(19) United States
(12) Patent Application Publication
(10) Pub. No.: US 2007/0056585 A1
(43) Pub. Date: Mar. 15, 2007

(54) Medicament dispenser

(76) Inventors: Michael Birsha Davies, Ware (GB); Mark Hedley, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/572,916

(22) PCT Filed: Sep. 21, 2004
(86) PCT No.: PCT/EP04/10654

§ 371 Date: Nov. 1, 2006

(30) Foreign Application Priority Data
Sep. 23, 2003 (GB) 03222284.1

(51) Int. Cl. B65D 83/06; A61M 15/00

(52) U.S. Cl. 128/203.15

(57) ABSTRACT

There is provided a medicament dispenser device comprising a housing including a dispensing outlet for insertion into a body cavity; a medicament discharge device moveably housed within the housing, the medicament discharge device comprising a medicament container for storing the medicament to be dispensed and a medicament dispensing mechanism for dispensing medicament from the container to the outlet; a user operable actuator moveable with respect to the medicament discharge device to apply an actuating force to the dispensing mechanism; and an actuation indicator responsive to application of the actuating force. A pre-load means is provided to the user operable actuator to prevent application of the actuating force to the dispensing mechanism and actuation indicator until a pre-determined threshold force is applied to the user operable actuator.

The present invention relates to a medicament dispenser including a medicament container having a dispensing mechanism actuable by an actuator. The dispenser includes an actuation counter.

It is well known to use a medicament dispenser to dispense medicament for administration to a patient (e.g. for inhalation to the lung or for application to the nasal cavity) and a wide variety of medicament dispensers have been developed for this purpose.

Known medicament dispensers include those in which, a medicament formulation is contained in a pressurized aerosol container and administered to a patient by means of an inhalation device comprising a tubular housing or sleeve in which the aerosol container is located and an outlet tube leading out of the tubular housing. Such inhalation devices are generally referred to as metered dose inhalers (MDIs). The aerosol containers used in such inhalation devices are designed to deliver a predetermined dose of medicament upon each actuation by means of an outlet valve member at one end which can be opened either by depressing the valve member while the container is held stationary or by depressing the container while the valve member is held stationary. In the use of such devices, the aerosol container is placed in the tubular housing with the outlet valve member of the container communicating via a support with the outlet tube, for example a nozzle or mouthpiece. In use, the patient holds the housing in a more or less upright condition and the mouthpiece or nozzle of the inhalation device is placed in the mouth or nose of the patient. The aerosol container is moved towards the support to dispense a dose of aerosol spray form medicament from the container, which is then inhaled by the patient.

Other known medicament dispensers include dry powder inhalation devices for the delivery of powder form medicament. In one aspect, such dispensers comprise a reservoir of powdered medicament from which doses are metered prior to or concurrent with the delivery process. The dispenser may be designed for active release whereby a ‘puff’ of gas or air is provided to the delivery position to assist in aerosolisation of the powder prior to or concurrent with the inhalation of the patient. Such devices are generally called active release dry powder inhalers (active DPIs). The source of the compressed gas or air is generally an aerosol container but can also be provided by another suitable means such as a pump or plunger mechanism.

Other known medicament dispensers include those in which fluid form medicament may be dispensed as a spray via a nozzle or orifice upon the application of user force to a pump form actuator. Such spray devices may be arranged to dispense a single dose or may alternatively be arranged with a reservoir containing several doses to be dispensed.

Other known medicaments include syringes for the delivery of injectable medicament to a patient. Syringes rely on puncturing of the patient's skin by a hollow needle through which the injectable medicament (in solution or suspension form) is delivered to the muscle or tissue of the patient by a plunger mechanism.

It may be appreciated that effective delivery of medicament to the patient using any of the different types of medicament dispensers described above requires some sort of actuation step, generally invoked by a manual user action on an actuator (e.g. depressing an MDI to open the valve and fire the dose; pump actuating a nasal pump to deliver liquid dose; or moving the plunger of a syringe to inject dose).

It is desirable that any particular medicament dispenser is configured to provide the patient with feedback relating either to how many doses of medicament have been delivered from the device or often more importantly, how many doses remain within the dispenser. Thus, various dose counters have been developed for use with different types of medicament delivery device. Both mechanical and electronic counters are known and also both analogue and digital count displays.

It may be appreciated that dose counters can be arranged to count in response to ‘release’ of medicament product, or more commonly to count in response to ‘actuation’ of the dispenser mechanism of the dispenser device. The Applicant has now however, realised that dose counting potentially gives rise to problems where ‘actuation’ of a user operable actuator of the dispenser is relied on to register the count.

One problem arises if the user operable actuator is moved in a slow or unpredictable manner such that the medicament is not effectively dispensed, but where the counter still registers an actuation count. This can be particularly significant where effective dispensing relies on manual patient invocation of a complete actuation step (e.g. depressing an MDI far enough to open the valve and fire the complete dose; pump actuating a nasal pump strongly enough to deliver the entire liquid dose as a suitable spray; or moving the plunger of a syringe sufficiently to inject the entire dose). By way of a solution to this problem, the medicament dispenser device herein includes a ‘commitment’ feature, which prevents actuation of the actuation, and hence registering of count, in the absence of the application of pre-determined, threshold force to a user operable actuator.

According to a first aspect of the invention there is provided a medicament dispenser device comprising

a housing including a dispensing outlet;

a medicament discharge device moveably housed within the housing, the medicament discharge device comprising a medicament container for storing the medicament to be dispensed and a medicament dispensing mechanism for dispensing medicament from the container to the outlet;

a user operable actuator moveable with respect to the medicament discharge device to apply an actuating force to the dispensing mechanism; and

an actuation indicator responsive to application of said actuating force,

wherein a pre-load means is provided to said user operable actuator to prevent application of the actuating force to the dispensing mechanism and actuation indicator until a pre-determined threshold force is applied to the user operable actuator.

In general terms, the present invention in accord with the first aspect is suitably embodied as an MDI, nasal pump or syringe type medicament dispenser device.

It may be appreciated that, in accord with present invention, the pre-load means acts such as to prevent transfer of user applied force to both the dispensing mechanism and actuation indicator until sufficient force to is applied to the user operable actuator to overcome the pre-determined threshold.

The medicament dispenser device has a housing, suitably including a dispensing outlet for insertion into a body cavity. The housing can take any suitable form, but is suitably arranged for ease of holding within the hand of a user.

The outlet may have any suitable form. In one aspect, it has the form of a mouthpiece for oral insertion and in another aspect it has the form of a nozzle for insertion into the nasal cavity of a patient.

The housing is shaped for receipt of a medicament discharge device that is moveable within the housing. In aspects, the medicament discharge device is partly or wholly housed within the housing for movement relative thereto.

The medicament discharge device comprises a medicament container for storing the medicament to be dispensed and a medicament dispensing mechanism for dispensing medicament from the container to the outlet for delivery to the patient.

The medicament discharge device may be arranged to have any suitable form, and in particular the medicament contained within the medicament container may have a variety of forms including dry powder, pressurized aerosol and liquid form.

In one aspect, the medicament dispenser device has the form of a metered dose inhaler (MDI). That is to say, the device is a medicament dispenser suitable for dispensing medicament in aerosol form. The medicament discharge device therefore comprises an aerosol medicament container suitable for containing a propellant-based aerosol medicament formulation. The aerosol medicament container is provided with a dispensing mechanism comprising a metering valve, for example a slide valve, for release of the aerosol form medicament formulation to the patient. The medicament aerosol container is generally designed to deliver a predetermined dose of medicament upon each actuation by means of the valve dispensing mechanism, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation.

In another aspect, the medicament dispenser device has the form of a pump dispenser for fluids, particularly a nasal pump. That is to say, the medicament discharge device is suitable for pump dispensing medicament in fluid form. The medicament discharge device therefore comprises a medicament container suitable for containing fluid medicament (formulation). The aerosol medicament container is provided with a dispensing mechanism comprising a pump.

Suitably, the fluid medicament discharge device has a longitudinal axis and comprises a container for storing the fluid to be dispensed and a compression pump having a suction inlet located within the container and a discharge tube extending along the longitudinal axis for dispensing fluid from the container. In use, the user operable actuator moves transversely with respect to the longitudinal axis of the fluid discharge device to apply a force to the container to move the container along the longitudinal axis so as to actuate the pump.

Suitably, the pump comprises a pre-compression pump, such as a VP3, VP7 or modifications, model manufactured by Valois SA. Typically, such pre-compression pumps are typically used with a bottle (glass or plastic) container capable of holding 8-50 ml of a formulation. Each spray will typically deliver 50-100 μl of such a formulation and the device is therefore capable of providing at least 100 metered doses.

In another aspect, the medicament dispenser device has the form of a syringe dispenser of fluid for injection. Known syringes rely on puncturing of the patient's skin by a hollow needle through which the injectable medicament is delivered to the muscle or tissue of the patient. The medicament container is therefore typically in the form of a barrel suitable for receipt of medicament (formulation) for injection and the dispensing mechanism comprises a plunger for plunge dispensing of the medicament (formulation) from the barrel to the hollow needle. The syringe contents may for example, be liquid, solutions, suspensions, particulates or in freeze-dried form. A retract or reset mechanism is typically provided for the plunger.

The medicament dispenser has a user operable actuator moveable with respect to the medicament discharge device to apply an actuating force to the dispensing mechanism.

The term user operable actuator means is meant to encompass such actuator means manually operable by action of the finger or thumb, or combinations thereof of a typical user (e.g. an adult or child patient).

In one aspect, the user operable actuator is moveable transversely with respect to a longitudinal axis defined by the medicament discharge device to apply an actuating force directly or indirectly to the medicament container. In alternative aspects, the user operable actuator may therefore contact the container or be coupled thereto to enable the necessary transfer of actuating force.

In one aspect, the user operable actuator is arranged to apply mechanical advantage. That is to say, the user operable actuator applies mechanical advantage to the user force to adjust (generally, to enhance or smooth) the actuating force experienced by the container. The mechanical advantage may in one aspect, be provided in either a uniform manner such as by a constant mechanical advantage enhancement, for example by a ratio of from 1.5:1 to 10:1 (enhanced force:initial force), more typically from 2:1 to 5:1. In another aspect, the mechanical advantage is applied in a non-constant manner such as progressive increase or progressive decrease of mechanical advantage over the applied force cycle. The exact profile of mechanical advantage variation may be readily determined by reference to the desired medicament dispensing profile and all relevant characteristics of the device and medicament (formulation) to be dispensed.

Suitably, the user operable operator has a form, which naturally gives rise to mechanical advantage such as a lever, cam or screw form.

In one aspect, the user operable actuator comprises at least one lever pivotally connected to part of the housing and arranged to transfer actuating force to the container (e.g. acting directly thereupon) so as to urge the container towards the dispensing outlet when the or each lever is moved by a user.

In another aspect, there are two opposing levers, each of which pivotally connect to part of the housing and may be arranged to act upon the medicament container so as to urge the medicament container towards the dispensing outlet when the two levers are squeezed together by a user.

Suitably, the user operable actuator acts on a transition piece connecting to a neck of the medicament container. Suitably, the transition piece is in the form of a collar.

In aspects, the or each lever may be pivotally supported at a lower end within the housing and the user operable actuator connects to a neck of the medicament container (e.g. formed as a collar thereto).

In another aspect, the user operable actuator comprises at least one lever slidably supported within the housing to apply an actuating force to the medicament container so as to move the container towards the dispensing outlet and actuate the dispensing mechanism.

The actuation indicator is responsive to application of the actuating force. The term ‘actuation indicator’ is used herein to mean any means for indicating, or in particular counting, when actuating force is provided to the dispensing mechanism.

The term ‘actuation’ is generally used to mean actuation of the dispensing mechanism device such that medicament is dispensed from the container.

Actuation indication may be based on detection of any actuation step, which results in actuating force being provided to the dispensing mechanism (i.e. a successful actuation step resulting from sufficient force being provided to overcome the predetermined threshold force).

The actuation indicator particularly includes means for registering and displaying dose count information to the patient. At a basic level, that information may simply relate to the fact that a successful actuation step has been detected, but more often the information relates to the number of doses delivered or remaining of medicament in the dispenser device. The information may be delayed in digital or analogue form, typically using standard count indicia (e.g. ‘999’to ‘000’ indicia count display). Embodiments involving either ‘counting up’ or ‘counting down’ in increments are envisaged.

For detection of a successful actuation step, the dispenser device may suitably comprise an actuation sensor. The actuation sensor is for example, sensitive to parameters selected from the group consisting of electro magnetic radiation, magnetic field, light, motion, temperature, pressure, sound, oxygen concentration, carbon dioxide concentration and moisture. The actuation sensor is arranged to sense the successful actuation of the dispenser. In one aspect, the actuation sensor is integral with the housing, for example moulded into a housing of the dispenser device or attached thereto. Alternatively, the actuation sensor is reversible attachable to the housing.

The actuation indicator may be associated mechanically or electronically with the actuation sensor(s), such that when the sensor detects actuation a signal is sent to the actuation indicator to record that a (part) dose has been dispensed.

In one aspect, the actuation indicator comprises a microprocessor. Suitably, the microprocessor performs operations on the data from any sensor and produces a signal output relating to the data or the outcome of an operation on the data.

Suitably, the actuation indicator is provided with or communicates with a visual display unit for display of the data. Preferably, the visual display unit displays the number of doses of medicament used or remaining within the container.

Suitably the doses are displayed numerically or by a series of coloured lights or by a monochrome bar graph.

A pre-load means is provided to the user operable actuator. The pre-load means acts such as to prevent application of the actuating force to the dispensing mechanism and actuation indicator until a pre-determined threshold force is applied to the user operable actuator. The pre-determined force may thus, be thought of as a ‘threshold’ or ‘barrier’ force which must first be overcome before actuation of the dispensing mechanism, and register of that actuation by the actuation indicator, can occur.

The quantum of pre-determined force that is to be overcome before actuation of the dispensing mechanism is enabled is selected according to various factors including characteristics of the dispensing mechanism, typical user profile, nature of the medicament (formulation) and the desired dispensing characteristics.

Typically, the pre-determined threshold force is in the range from 5 to 30 N, more typically from 10 to 25 N. That is to say, typically from 5 to 30 N, more typically from 10 to 25N of force must be applied to the user operable actuator before actuation of the dispensing mechanism is enabled. Such values tend to correspond to a force which prevents a suitable ‘barrier force’ to a weak, nondescript or unintended user (e.g. finger) movement whilst readily being overcome by the determined finger (or thumb) action of a user. It will be appreciated that if the device is designed for use by a child or elderly patient it may have a lower pre-determined force than that designed for adult usage.

In accordance with a first embodiment of the invention the pre-load means is physically interposed between the user operable actuator and the medicament container.

In which case, the pre-load means may comprise of a step formed on the medicament container that must be ridden over by the user operable actuator before the dispensing mechanism can be actuated wherein the step is over-ridden when the pre-determined threshold force is applied to the user operable actuator.

Alternatively, the pre-load means may comprise of a step formed on the or each user operable actuator that must be ridden over by the medicament container before the dispensing mechanism can be actuated wherein the step is over-ridden when the pre-determined threshold force is applied to the user operable actuator.

In yet a further alternative, the pre-load means may comprise of at least one detent formed on one of the medicament container or the user operable actuator and a recess formed on the other of the medicament container or the user operable actuator wherein the or each detent is able to ride out of the recess with which it is engaged when the pre-determined threshold force is applied to the user operable actuator.

In accordance with a second embodiment of the invention the pre-load means is interposed between the housing and the medicament container.

In which case, the pre-load means may comprise of one or more detents formed on the medicament container for engagement with part of the housing, the or all of the detents being disengageable from the housing when the pre-determined threshold force is applied to the user operable actuator so as to allow the dispensing mechanism to be actuated.

Alternatively, the pre-load means may comprise of one or more detents formed on the housing for engagement with part of the medicament container, the or all of the detents being disengageable from the medicament container when the predetermined threshold force is applied to the user operable actuator so as to allow the dispensing mechanism to be actuated.

In accordance with a third embodiment of the invention the pre-load means is interposed between the housing and the user operable actuator.

In which case, the pre-load means may comprise of at least one detent formed on the housing for engagement with the user operable actuator, the or all of the detents being disengageable from the user operable actuator when the pre-determined threshold force is applied to the user operable actuator so as to allow the dispensing mechanism to be actuated.

Alternatively, the pre-load means may comprise of at least one detent formed on the user operable actuator for engagement with part of the housing, the or all of the detents being disengageable from the housing when the pre-determined threshold force is applied to the user operable actuator so as to allow the dispensing mechanism to be actuated.

As yet a further alternative, the pre-load means (e.g. comprised at a finger operable means) defines a variable mechanical ratio such that until the pre-determined force is applied to the or each user operable actuator (e.g. a lever) no significant force is transferred to the container along the longitudinal axis. The variable mechanical ratio is suitably defined by the profile of interaction of a surface of the user operable actuator with a follower element provided to the container or a fitting provided thereto (e.g. a collar).

In one aspect, the variable mechanical ratio defines a ‘two step’ profile characterized by an initial ‘high force’ (e.g. high gradient) profile (defining the pre-load force, to be overcome) and a subsequent ‘low force’ (e.g. low gradient) profile.

In one particular aspect, the ‘high force’ and ‘low force’ profiles are linear (i.e. straight lines) and have a sharp break point therebetween.

In another particular aspect, the ‘high force’ and ‘low force’ profiles are curved and have a smooth/gradual break point therebetween.

In a preferred aspect, the ‘high force’ and ‘low force’ profiles have part-circle profile forms (e.g. as would be defined be overlapping circles of different radii and different centres) and have a smooth/gradual break point therebetween.

In another aspect, the pre-load means comprises a spring interposed between the user operable actuator and the medicament container, the spring being used to urge the medicament container in a particular sense (i.e. towards or away from) relative to the dispensing mechanism.

In which case the spring may be compressed by movement of the user operable actuator until the pre-determined threshold force is applied (i.e. by a combination of user-applied force and stored spring urging force), at which point the threshold of the preload means used to prevent actuation of the dispensing mechanism is overcome by the force being applied to the medicament container such that the container moves so as to actuate the dispensing mechanism.

Suitably, the medicament dispenser device is additionally provided with force modifying means for modifying the force applied to the container. That is to say, means for modifying the force applied to (and therefore, ultimately acting on) the container compared to that force directly applied to the user operable actuator by the user.

In one aspect, the force modifying means acts such as to amplify the force applied (i.e. it comprises force amplifying means). The amplification may be provided in either a uniform manner such as by a constant amplification, for example by a ratio of from 1.5:1 to 10:1 (amplified force: initial force; i.e. degree of amplification of from 1.5 to 10), more typically from 2:1 to 5:1. In another aspect, the amplification is applied in a non-constant manner such as progressive increase or progressive decrease of mechanical advantage over the applied force cycle.

The exact profile of force modification may be readily determined by reference to the desired dispensing profile and all relevant characteristics of the device and medicament (formulation) to be dispensed.

The force modifying means may in one aspect, be integral with the user operable actuator. In this aspect, the force modifying means may comprise an aspect of the user operable actuator shaped to give rise to a mechanical advantage (e.g. a lever, cam or screw feature).

In another aspect, the force modifying means is located non-integral with the user operable actuator, and typically between the user operable actuator and the medicament container. Again this aspect, the force modifying means may comprise an aspect of the user operable actuator shaped to give rise to a mechanical advantage (e.g. a lever, cam or screw feature).

In one aspect, the force modifying means only acts (i.e. only acts to modify the user applied force) once the pre-determined threshold force has been overcome. In preferred aspects, the modifying force acts such that once the pre-determined threshold force has been overcome the force applied to the dispensing mechanism is either relatively constant or increases on a relatively constant basis.

In one particular aspect, the force modifying means additionally comprises a stop feature, which acts to stop actuating force being applied to the dispensing mechanism once either a particular maximum force is reached or more typically, once the user operable actuator has been moved a particular distance relative to the medicament discharge device. In one aspect, the stop functions to prevent excess force being applied to the dispensing mechanism.

Embodiments are envisaged in which the medicament discharge device is reversibly removable from the housing of the medicament dispenser device. In such embodiments the medicament dispenser device comprises a housing assembly and medicament discharge device receivable thereby.

According to another aspect of the present invention there is therefore provided a housing assembly for reversible receipt of a medicament discharge device, said medicament discharge device comprising a medicament container for storing the medicament to be dispensed and a medicament dispensing mechanism for dispensing medicament from the container, the housing assembly comprising

a housing including a dispensing outlet for insertion into a body cavity;

a user operable actuator moveable with respect to the medicament discharge device to apply an actuating force to the dispensing mechanism; and

an actuation indicator responsive to application of said actuating force,

wherein a pre-load means is provided to said user operable actuator to prevent application of the actuating force to the dispensing mechanism and actuation indicator until a pre-determined threshold force is applied to the user operable actuator.

According to a still further aspect of the present invention there is provided a kit of parts comprising a housing assembly as described above and a medicament device receivable thereby. The medicament discharge device comprises a medicament container for storing the medicament to be dispensed and a medicament dispensing mechanism for dispensing medicament from the container to the outlet of the housing assembly.

In variations herein, the present invention may be embodied as an ‘active’ (e.g. active DPI) type dispenser in which actuation of airflow generating means, which provides airflow to assist in aerosolising the medicament dose, is prevented until application of the threshold force to the user operable actuator.

Thus, according to another aspect of the invention there is provided a medicament dispenser device comprising

a housing including a dispensing outlet for insertion into a body cavity;

within the housing, a medicament release device, the medicament release device comprising a medicament container for storing the medicament to be dispensed and a medicament release mechanism for releasing medicament from the container to a release position within the housing;

an airflow generator moveably housed within the housing, said airflow generator means capable on actuation, of providing airflow to said release position for aerosolising said released medicament;

a user operable actuator moveable to apply an actuating force to the airflow generator; and

an actuation indicator responsive to application of said actuating force,

wherein a pre-load means is provided to said user operable actuator to prevent application of the actuating force to the airflow generator and actuation indicator until a pre-determined threshold force is applied to the user operable actuator.

In this aspect, the airflow generator may comprise any suitable source of airflow sufficient to assist in aerosolising the released medicament at the release position. In one aspect, the airflow generator is a source of compressed gas or air that is generally comprised within an aerosol container. In another aspect, the airflow generator comprises a pump or plunger mechanism for airflow generation.

Also, in this aspect it will be appreciated that the term ‘actuation indicator’ is used to mean any means for indicating, or in particular counting, when the actuating force is provided to the airflow generator. The term ‘actuation’ is thus in this aspect, used to mean actuation of the airflow generator such that medicament is aerosolised for dispensing thereof.

Further, in this aspect the medicament release device is suitably of the reservoir, dry powder type. That is to say, the device comprises a reservoir form container pack suitable for containing multiple (un-metered doses) of medicament product in dry powder form and includes means for metering medicament dose from the reservoir to the release position. The metering means may for example comprise a metering cup, which is movable from a first position where the cup may be filled with medicament from the reservoir to a second position where the metered medicament dose is made available to the patient for inhalation.

### DESCRIPTION OF THE DRAWINGS

The invention will now be described further with reference to the accompanying drawings in which:

FIG. 1 is a sectional side view of a first medicament dispenser in accord with the present invention;

FIG. 2 is a perspective view of a collar for use in the first medicament dispenser of FIG. 1;

FIG. 3 is a perspective view of an actuating lever for use in the first medicament dispenser of FIG. 1;

FIG. 4 is a sectional side view of a second medicament dispenser in accord with the present invention;

FIG. 5 is a perspective view from above of the second medicament dispenser of FIG. 4 absent its housing; and

FIG. 6 is a sectional side view of a detail of the second medicament dispenser of FIG. 4 illustrating the passage of air flow on actuation thereof;

FIGS. 7a and 7b, each show a perspective view of an alternative collar arrangement for use with the first medicament dispenser of FIG. 1 or second medicament dispenser of FIG. 4;

FIG. 8a is a perspective side view of a third medicament dispenser in accord with the present invention;

FIG. 8b is a sectional side view of the third medicament dispenser of FIG. 8a;

FIGS. 9a and 9b respectively show sectional side views of details of the actuation of the third medicament dispenser of FIGS. 8a and 8b;

FIG. 10 shows a perspective, cut-away view of a detail of the counter of the third medicament dispenser of FIGS. 8a and 8b;

FIG. 11 is a perspective side view of a fourth medicament dispenser in accord with the present invention;

FIG. 12 is a perspective side view of the actuation of the fourth medicament dispenser of FIG. 11; and

FIG. 13 is a perspective view of the counter of the fourth medicament dispenser of FIGS. 11 and 12.

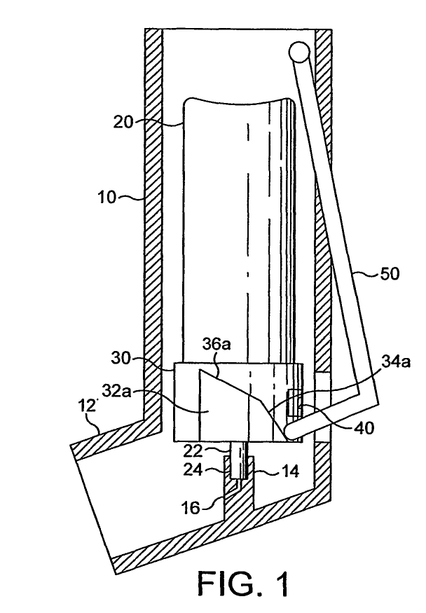

FIG. 1 shows a medicament dispenser herein in the form of a metered dose inhaler for the delivery of medicament for inhalation by a patient. The inhaler comprises a tubular housing 10 in which an aerosol container 20 is located. A dispensing outlet 12 leads laterally from the closed end of the housing 10. In the embodiment illustrated, the outlet 12 is in the form of a mouthpiece intended for insertion into the mouth of the patient but it may, if desired, be designed as a nozzle for insertion into the patient's nostril.

The aerosol container 20 has a valve dispensing mechanism 22 in the form of a slide valve. In variations, the dispensing mechanism may comprise a pump dispenser. Valve stem 24 connects with a support 14. The support 14 is provided with an outlet passage 16 enabling dispensed dose to pass through to the dispensing outlet 12. It will be appreciated that dispensing of the dose requires the aerosol container 20 to be depressed to actuate the slide valve dispensing mechanism 22 and dispense medicament into the outlet 12 from which it can be inhaled by a patient.

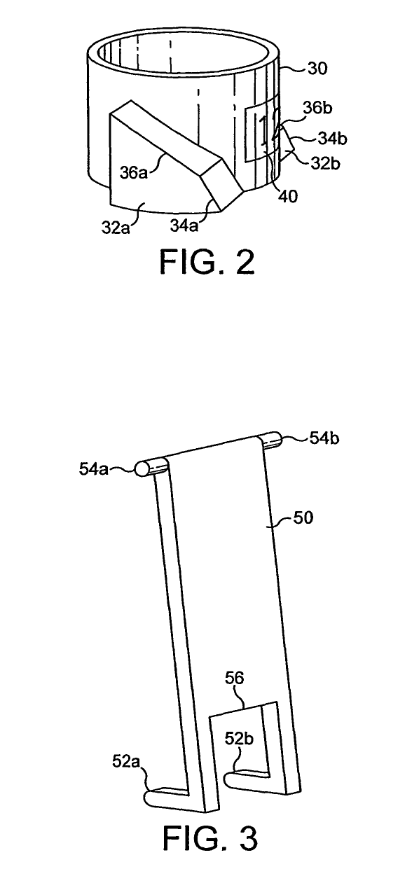

The neck (i.e. at the valve end) of the aerosol container 20 is provided with a collar 30, which is typically fixed attached thereto (e.g. by a joining method as described in published PCT application no. WO 01/28887). A perspective view of the collar is provided at FIG. 2. Within the collar 30, there is provided an actuation indicator 40, which is actuable in response to actuation of the dispensing mechanism 22 by depression of the aerosol container 20 within the housing 10. The actuation indicator 40 may comprise mechanical or electronic components and is suitably of a type known in the art.

The collar 30 may be seen to be provided at either side with wings 32a, 32b (only one side visible in FIG. 1) wherein each wing 32a, 32b is shaped to define a first steeply inclined ramp surface 34a, 34b and a second ramp surface 36a, 36b of shallow incline. The wing 32a, 32b surfaces are designed to act as guides for follower ends 52a, 52b (only one side visible in FIG. 1) of lever actuator arm 50. As will become apparent from the further description provided below, the interaction between followers 52a, 52b and wing 32a, 32b surfaces provides the actuation ‘commitment’ feature in accord with the present invention.

A perspective view of the lever actuator arm 50 is shown at FIG. 3. The arm 50 may be seen to pivotally mount to the housing 10 at pivot points 54a, 54b (only only visible in FIG. 1). When the lever arm 50 is at rest the follower ends 52a, 52b abut steeply inclined ramp surfaces 34a, 34b of the wings 32a, 32b provided to the collar. Cut-away portion 56 allows the actuation indicator 40 to be visible to the user when the actuator arm 50 is mounted to the housing 10.

In a first stage of an actuation movement, the patient pushes the lever arm 50 towards the housing 10. Initially, a pre-load resistive force is experienced as a result of the follower ends 52a, 52b of the lever arm 50 being guided by the first steeply inclined ramp surfaces 34a, 34b. A pre-load resistive force, the magnitude of which is determined by the gradient and length of the first ramp surfaces 34a, 34b, initially prevents actuation of the dispensing mechanism 22 and actuation indicator 40. The patient must therefore apply more force to the lever arm 50 until sufficient force is provided to cause the follower ends 52a, 52b to overcome the pre-load resistive force of the first steeply inclined ramp surfaces 34a, 34b. Once the ‘commitment’ threshold force is so overcome, the second, more shallow, ramp surfaces 36a, 36b are experienced. These provide minimal further resistive force and the follower ends 52a, 52b therefore ride quickly over the shallow ramp surfaces 36a, 36b to enable rapid transfer of downward force to the collar 30 and hence to the aerosol container 20. The container 20 moves downward relative to the housing 10 to actuate the dispensing mechanism 22 to release medicament formulation through the outlet 12 and also trigger registration of that actuation by the actuation indicator 40.

In variations of the embodiment of FIGS. 1 to 3, the dual ramp surfaces may be substituted by other means of providing suitable threshold ‘commitment’ force. In particular, the first ramp surfaces may be replaced by a détente feature (e.g. a nib on a guide surface) that must be physically overcome before actuation is enabled.

In further variations of the embodiment of FIGS. 1 to 3, the metered dose inhaler form is substituted by a pump-form dispenser or a pump-form syringe, both of which rely on actuating movement of a medicament container relative to a housing for delivery of medicament therefrom.

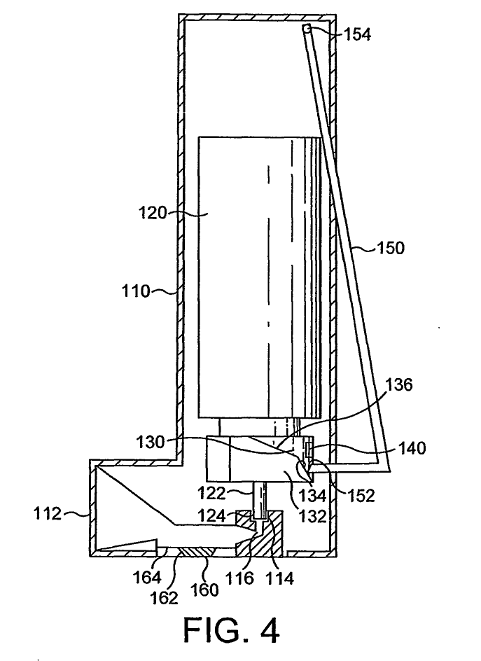

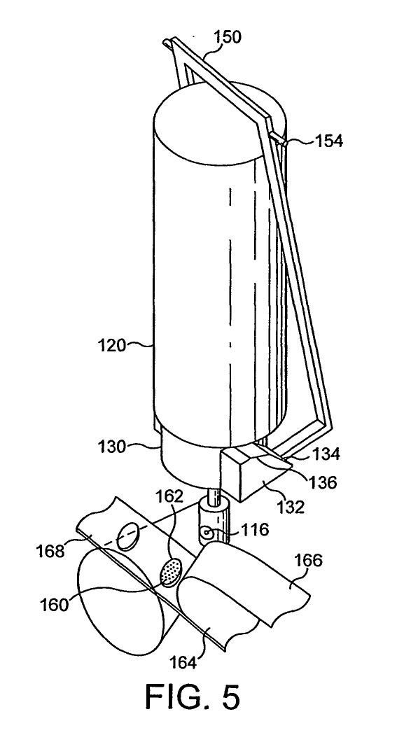

FIG. 4 shows a medicament dispenser herein in the form of an active dry powder inhaler (DPI) for the aerosolised delivery of dry powder medicament for inhalation by a patient. FIG. 5 shows the dispenser of FIG. 4, but absent its housing. The inhaler comprises a tubular housing 110 in which an aerosol container 120 containing compressed air is located. A dispensing outlet 112 leads laterally from the closed end of the housing 110. In the embodiment illustrated, the outlet 112 is in the form of a mouthpiece intended for insertion into the mouth of the patient but it may, if desired, be designed as a nozzle for insertion into the patient's nostril.

The aerosol container 120 has a valve dispensing mechanism 122 in the form of a slide valve. In variations, the dispensing mechanism may comprise a pump dispenser. Valve stem 124 connects with a support 114. The support 114 is provided with an outlet passage 116 enabling a puff of air to pass through to the medicament dose 160 held within the opened pocket 162 of a blister pack 164 for aerosolisation thereof. It will be appreciated that dispensing of the dose 160 from the opened pocket 162 requires the aerosol container 120 to be depressed to actuate the slide valve dispensing mechanism 122 and dispense a puff of air to the dose 160, which is aerosolised thereby and may be carried by the air flow in aerosolised form into the outlet 112 from which it can be inhaled by a patient.

As best seen in FIG. 5, the blister pack 164 is conveniently in the form of an elongate strip comprising a lid foil 166 peelably separable from a base foil 168 to reveal an open pocket 162 from which medicament dose 160 in powder form may be accessed.

The neck (i.e. at the valve end) of the aerosol container 120 is provided with a collar 130, which is typically fixed attached thereto (e.g. by a joining method as described in published PCT application no. WO 01/28887). The collar 130 has the general form of that shown previously described in respect of the first medicament dispenser and shown in FIG. 2. Within the collar 130, there is provided an actuation indicator 140, which is actuable in response to actuation of the dispensing mechanism 122 by depression of the aerosol container 120 within the housing 110. The actuation indicator 140 may comprise mechanical or electronic components and is suitably of a type known in the art.

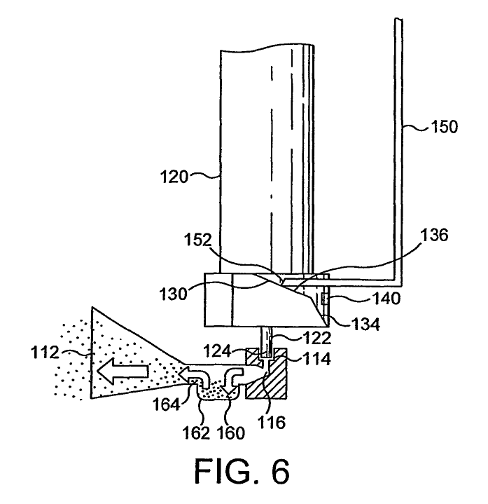

The collar 130 may be seen to be provided at either side with wings 132 (only one side visible in FIGS. 4 to 6) wherein each wing 132 is shaped to define a first steeply inclined ramp surface 134 and a second ramp surface 136 of shallow incline. The wing 132 surfaces are designed to act as guides for follower ends 152 (only one side visible in FIGS. 4 and 6) of lever actuator arm 150. As will become apparent from the further description provided below, the interaction between followers 152 and wing 132 surfaces provides the actuation ‘commitment’ feature in accord with the present invention.

A perspective view of the lever actuator arm 150 is shown at FIG. 3 (i.e. it has the same form as that as used in respect of the first medicament dispenser). The arm 150 pivotally mounts to the housing 110 at pivot points 154 (only only visible in FIGS. 4 to 6). When the lever arm 150 is at rest the follower ends 152 abut steeply inclined ramp surfaces 134 of the wings 132 provided to the collar. Cut-away portion (see FIG. 3) allows the actuation indicator 140 to be visible to the user when the actuator arm 150 is mounted to the housing 110.

In a first stage of an actuation movement, the patient pushes the lever arm 150 towards the housing 110. Initially, a pre-load resistive force is experienced as a result of the follower ends 152 of the lever arm 150 being guided by the first steeply inclined ramp surfaces 134. A pre-load resistive force, the magnitude of which is determined by the gradient and length of the first ramp surfaces 134 initially prevents actuation of the dispensing mechanism 122 and actuation indicator 140. The patient must therefore apply more force to the lever arm 150 until sufficient force is provided to cause the follower ends 152 to overcome the pre-load resistive force of the first steeply inclined ramp surfaces 134. Once the ‘commitment’ threshold force is so overcome, the second, more shallow, ramp surfaces 136 are experienced. These provide minimal further resistive force and the follower ends 152 therefore ride quickly over the shallow ramp surfaces 136 to enable rapid transfer of downward force to the collar 130 and hence to the aerosol container 120. The container 120 moves downward relative to the housing 110 to actuate the dispensing mechanism 122 to a puff or air to aerosolise the medicament dose 160 in the open pocket 162 for dispensing through the outlet 112. Actuation also triggers a count by the actuation indicator 140.

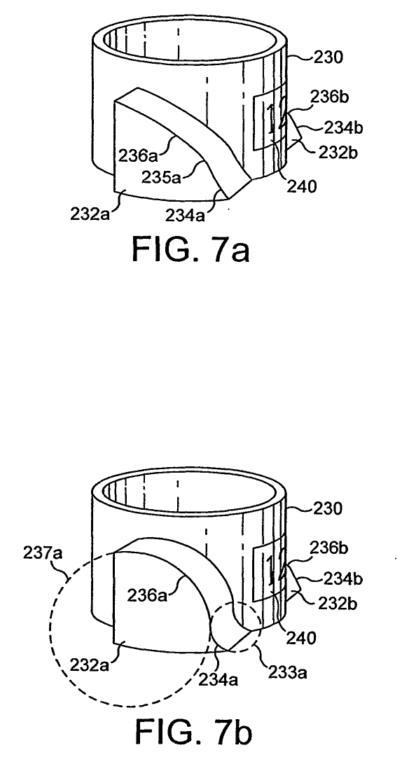

The form of collar 30, 130 used in the first and second medicament dispenser devices of FIGS. 1 to 3 and 4 to 6 respectively may varied, for example as shown in FIGS. 7a and 7b.

The profile defined by the ramps 234a, 234b and 236a, 236b of the wings 232a, 232b of the collar 230 and counter of FIG. 7a is that of an initial curved ‘high force’ (i.e. high gradient) profile 234a, 234b (defining the pre-load force, to be overcome) and a subsequent curved ‘lower force’ (i.e. lower gradient) profile 236a, 236b with a relatively smooth/gradual break point 235a (235b not visible) therebetween.

The profile defined by the ramps 234a, 234b and 236a, 236b of the wings 232a, 232b of the collar 230 and counter of FIG. 7b is that of an initial part-circle ‘high force’ (i.e. high gradient) profile 234a, 234b (defining the pre-load force, to be overcome) and a subsequent part-circle ‘lower force’ (i.e. lower gradient) profile 236a, 236b with a relatively smooth/gradual break point 235a (235b not visible) therebetween. In more detail, the ‘high force’ 234a, 234b and ‘low force’ 236a, 236b profiles may be seen to have profile forms as would be defined by overlapping circles 233a, 237a (233b and 237b not shown) of different radii and different centre points (illustrated schematically, in outline only).

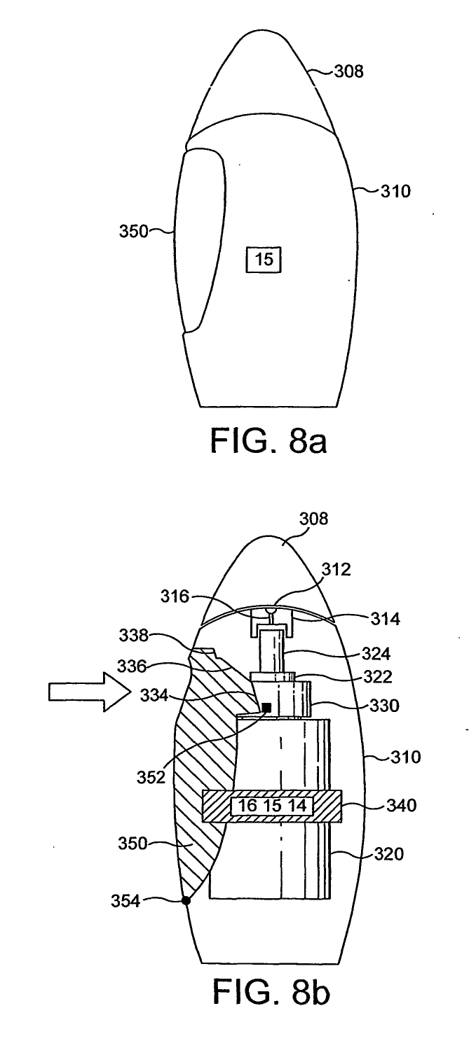

FIGS. 8a and 8b show a third medicament dispenser herein in the form of a pump dispenser for the delivery of fluid medicament to the nasal cavity of a patient. The inhaler comprises a housing 310, in which a container for fluid 320 is located. The housing 310 is provided with a cap 308. A dispensing outlet 312 in the form of a nozzle locates at one end of the housing 310.

The container 320 has a pump dispensing mechanism 322 in the form of a compression pump. Pump stem 324 connects with a support 314. The support 314 is provided with an outlet passage 316 enabling dispensed fluid dose to pass through to the dispensing outlet 312. It will be appreciated that dispensing of the dose requires the aerosol container 320 to be depressed to actuate the compression pump dispensing mechanism 322 and dispense fluid medicament to the outlet 312 from which it can be delivered to the nasal cavity of a patient.

The neck (i.e. at the valve end) of the container 320 is provided with a collar 330, which is typically fixed attached thereto (e.g. by a joining method as described in published PCT application no. WO 01/28887). Also provided to the container is actuation indicator 340, which is actuable in response to actuation of the dispensing mechanism 322 by depression of the container 320 within the housing 310. The actuation indicator 340, as shown comprises mechanical components.

The collar 330 is provided at either side (only one side visible) with a follower 352 that interacts with the ramped surface of lever actuator 350. The lever 350 mounts pivotally at pivot point 354 to the housing 310, and spring 356 biases the lever 350 to the non-actuated position. The ramped surface of the lever 350 may be seen to define a first steeply inclined ramp surface 334; a second ramp surface 336 of shallow incline; and a stop 338. The ramp surfaces 334, 336 are designed to act as guides for follower 352 of the collar 330.

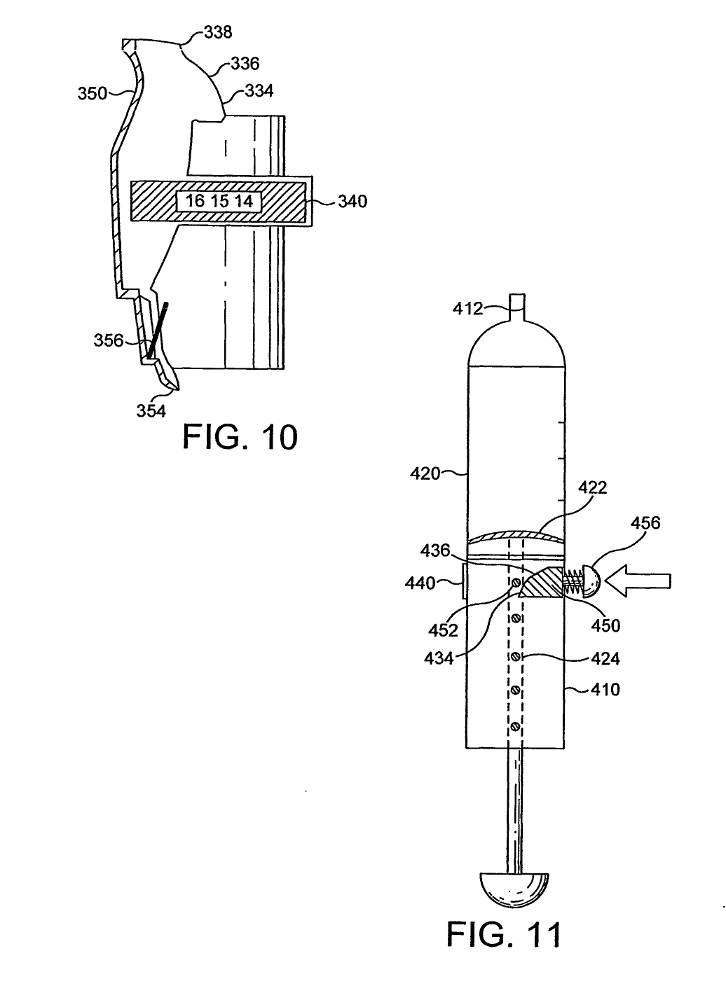

As may be seen in FIG. 10, the actuation indicator 340 is actuated in response to the lever 350 being brought into actuating contact therewith. Such actuating contact is only enabled once a ‘commitment’ threshold force has been overcome to actuate pump dispensing of fluid form medicament. As will become apparent from the further description provided below, the interaction between follower 352 and ramped surfaces 334, 336 of the lever 350 provides the actuation ‘commitment’ feature in accord with the present invention.

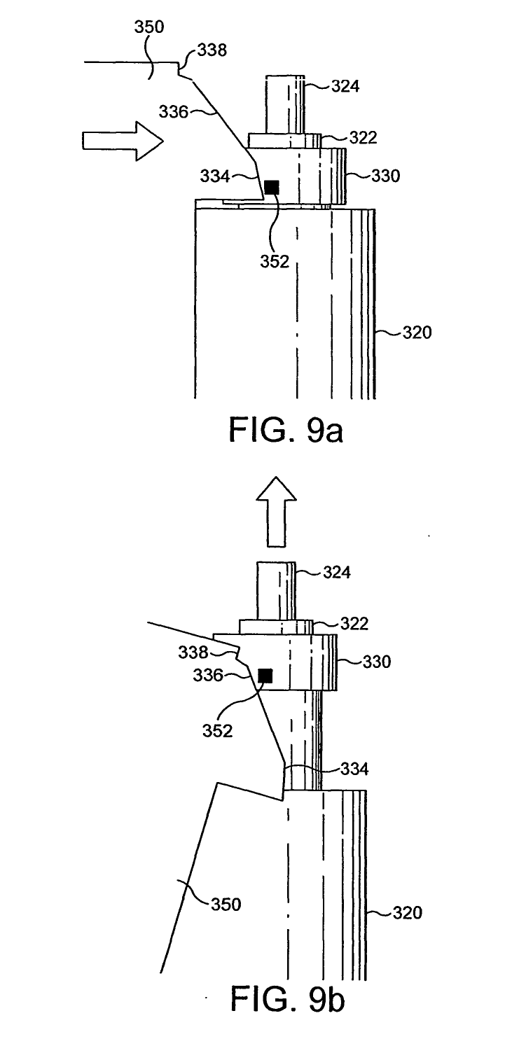

FIG. 9a shows the lever 350 in a non-actuated position and FIG. 9b shows the lever 350 late on in an actuating movement.

In a first stage of an actuation movement, the patient pushes the lever arm 350 laterally towards the housing 310. Initially, a pre-load resistive force is experienced as a result of the follower 352 of the collar 330 being guided by the first steeply inclined ramp surface 334. A pre-load resistive force, the magnitude of which is determined by the gradient and length of the first ramp surface 334, initially prevents actuation of the dispensing mechanism 322 and actuation indicator 340. The patient must therefore apply more force to the lever arm 350 until sufficient force is provided to cause the follower 352 to overcome the pre-load resistive force of the first steeply inclined ramp surface 334. Once the ‘commitment’ threshold force is so overcome, the second, more shallow, ramp surface 336 is experienced. This provides minimal further resistive force and the follower 352 therefore rides quickly over the shallow ramp surface 336 to enable rapid transfer of downward force to the collar 330 and hence to the pump dispenser 322. The pump dispenser 322 moves upwards relative to the housing 310 to actuate the pumped release of fluid form medicament formulation through the outlet 312. The lever 350 is also brought into actuating contact with the actuation indicator 340 thereby triggering a count of that actuation. Any overshoot of lever 350 is prevented by the interaction of follower 352 with stop 338 at the end of the stroke.

FIG. 11 shows a fourth medicament dispenser herein in the form of a syringe for the injected delivery of fluid medicament to a patient. The syringe comprises a barrel 410 defining a container for fluid 420. A dispensing outlet 412 in the form of a needle-receiving aperture locates at one end of the container 420.

The container 420 is provided with a dispensing mechanism in the form of a plunger 422. It will be appreciated that dispensing of the fluid medicament from the container requires the plunger 422 to be plunged into the container 420 to force plunged dispensing of fluid medicament to the outlet 412 from which it can be delivered via injection to the body of a patient.

The stem 424 of plunger 422 is provided with a series of follower pegs 452 (only one labelled) arranged to interact with the ramped surface of sprung lever actuator 450. The lever 450 mounts laterally at mounting point 454 to the housing 410, and spring 456 biases the lever 450 to the non-actuated position. Button 458 enables sprung return of the lever 450. The ramped surface of the lever 450 may be seen to define a first steeply inclined ramp surface 434; and a second ramp surface 436 of shallow incline. The ramp surfaces 434, 436 are designed to interact with follower pegs 452 of the plunger 422.

As best seen in FIG. 13, the housing 410 is also provided with actuation indicator 440, which is actuable in response to an actuating movement of the plunger 422 that brings a follower peg 452 into actuating contact therewith. Such actuating contact is only enabled once a ‘commitment’ threshold force has been overcome to actuate plunged dispensing of fluid form medicament. As will become apparent from the further description provided below, the interaction between follower 452 and ramped surfaces 434, 436 of the lever 450 provides the actuation ‘commitment’ feature in accord with the present invention.

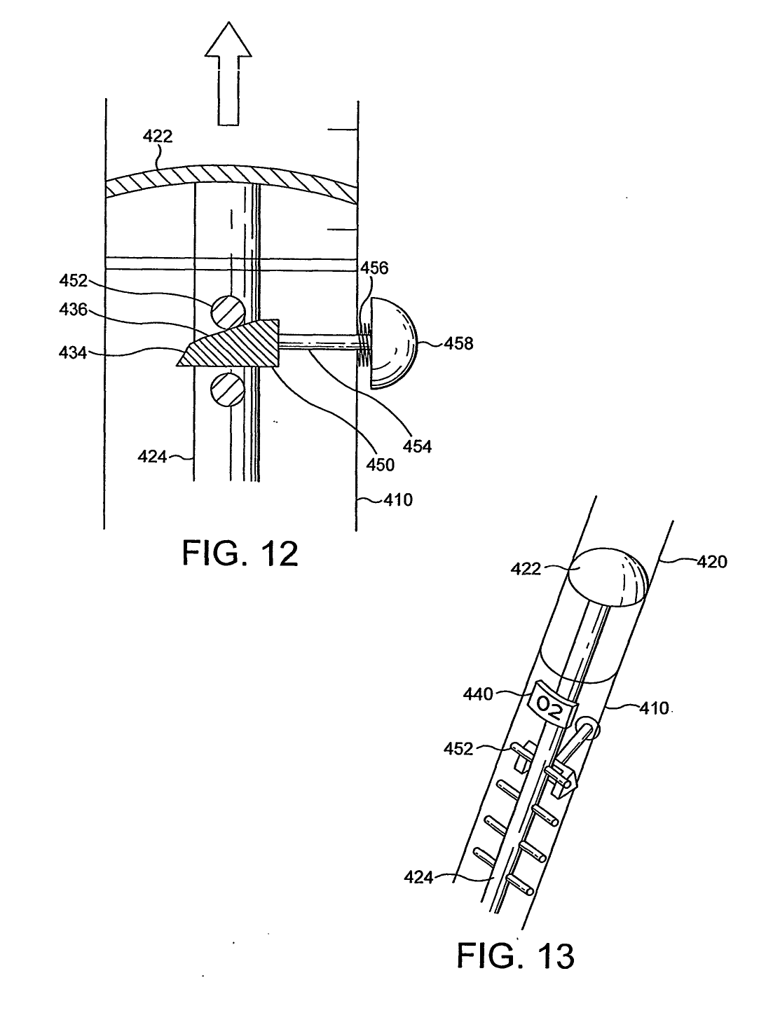

FIG. 11 shows the lever 450 in a non-actuated position and FIG. 12 shows the lever 450 late on in an actuating movement.

In a first stage of an actuation movement, the patient pushes the plunger 422 towards the container 420. Initially, a pre-load resistive force is experienced as a result of follower peg 452 of the plunger stem experiencing the first steeply inclined ramp surface 434. A pre-load resistive force, the magnitude of which is determined by the gradient and length of the first ramp surface 434, initially prevents further movement of the plunger 422 and hence also any actuating movement of the actuation indicator 440. The patient must therefore apply more force to the plunger 422 until sufficient force is provided to cause the follower 452 to overcome the pre-load resistive force of the first steeply inclined ramp surface 434. Once the ‘commitment’ threshold force is so overcome, the second, more shallow, ramp surface 436 is experienced. This provides minimal further resistive force and the follower 452 therefore rides quickly over the shallow ramp surface 436 to enable rapid transfer of force to the fluid contents of the container 420. The plunger 422 moves towards the container 420 to actuate the plunged release of fluid form medicament formulation through the outlet 412. The follower peg 452 is also brought into actuating contact with the actuation indicator 440 thereby triggering a count of that actuation.

It may be appreciated that any of the parts of the dispenser or actuator which contact the medicament may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

The medicament dispenser of the invention is in one aspect, suitable for dispensing medicament, particularly for the treatment of respiratory disorders such as asthma, chronic obstructive pulmonary disease (COPD) and rhinitis.

In another aspect, the medicament dispenser device herein is suitable for dispensing medicament for the treatment of a condition requiring treatment by the systemic circulation of medicament, for example migraine, diabetes, pain relief e.g. inhaled morphine.

Administration of medicament may be indicated for the treatment of mild, moderate or severe acute or chronic symptoms or for prophylactic treatment. It will be appreciated that the precise dose administered will depend on the age and condition of the patient, the particular medicament used and the frequency of administration and will ultimately be at the discretion of the attendant physician. Embodiments are envisaged in which combinations of medicaments are employed.

Appropriate medicaments are selected from, for example, analgesics, e.g., codeine, dihydromorphine, ergotamine, fentanyl or morphine; anginal preparations, e.g., diltiazem; antiallergics, e.g., cromoglycate (eg as the sodium salt), ketotifen or nedocromil (eg as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (eg as the dipropionate ester), fluticasone (eg as the propionate ester), flunisolide, budesonide, rofleponide, mometasone (eg as the furoate ester), ciclesonide, triamcinolone (eg as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl)ester or 6α,9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (eg as free base or sulphate), salmeterol (eg as xinafoate), ephedrine, adrenaline, fenoterol (eg as hydrobromide), formoterol (eg as fumarate), isoprenaline, metaproterenol, phenylephrine, phenylpropanolamine, pirbuterol (eg as acetate), reproterol (eg as hydrochloride), rimiterol, terbutaline (eg as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; PDE4 inhibitors eg cilomilast or roflumilast; leukotriene antagonists eg montelukast, pranlukast and zafirlukast; [adenosine 2a agonists, eg 2R,3R,4S,5R-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]*; [α4 integrin inhibitors eg (2S)-3-[4-({[4-(aminocarbonyl)-1-piperidinyl]carbonyl}oxy)phenyl]-2-[((2S)-4-methyl-2{[2-(2-methylphenoxy) acetyl]amino}pentanoyl)amino]propanoic acid (e.g as free acid or potassium salt)]*, diuretics, e.g., amiloride; anticholinergics, e.g., ipratropium (eg as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Particularly suitable medicament active components for the treatment of respiratory disorders are selected from those having either bronchodilator or anti-inflammatory action. The bronchodilator is suitably a beta-agonist, particularly a long-acting beta-agonist (LABA). Suitable bronchodilators include salbutamol (e.g., as the free base or the sulphate salt), salmeterol (e.g., as the xinafoate salt) and formoterol (eg as the fumarate salt). The anti-inflammatory is suitably an anti-inflammatory steroid. Suitably anti-inflammatory compounds include a beclomethasone ester (e.g., the dipropionate), a fluticasone ester (e.g., the propionate) or budesonide or any salt or solvate thereof.

In one aspect, the medicament is a glucocorticoid compound, which has anti-inflammatory properties. One suitable glucocorticoid compound has the chemical name: 6α,9α-Difluoro-17α-(1-oxopropoxy)-11β-hydroxy-16α-methyl-3-oxo-androsta-1,4-diene-17β-carbothioic acid S-fluoromethyl ester (fluticasone propionate). Another suitable glucocorticoid compound has the chemical name: 6α,9α-difluoro-17α-[(2-furanylcarbonyl)oxy]-11β-hydroxy-16α-methyl-3-oxo-androsta-1,4-diene-17β-carbothioic acid S-fluoromethyl ester. A further suitable glucocorticoid compound has the chemical name: 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

Other suitable anti-inflammatory compounds include NSAIDs e.g. PDE4 inhibitors, leukotriene antagonists, iNOS inhibitors, tryptase and elastase inhibitors, beta-2 integrin antagonists and adenosine 2a agonists.

The medicament is in one aspect, formulated as a combination product comprising plural medicament active components. Preferred components of combinations of active ingredients contain a bronchodilator in combination with an anti-inflammatory. The bronchodilator is suitably a beta-agonist, particularly a long-acting beta-agonist (LABA). The anti-inflammatory is suitably an anti-inflammatory steroid. One preferred combination of components comprises fluticasone propionate and salmeterol, or any salt or solvate thereof (particularly the xinafoate salt). A further combination of components of particular interest is budesonide and formoterol or any salt or solvate thereof (e.g. formoterol as the fumarate salt).

The medicament dispenser is one aspect, arranged to be a metered dose inhaler (MDI) type dispenser suitable for dispensing medicament in aerosol form.

Aerosol formulations suitable for use with metered dose inhaler (MDI) dispensers typically comprise a propellant. Suitable propellants include P11, P114 and P12, and the CFC-free hydrofluoroalkane propellants HFA-134a and HFA-227.

The MDI aerosol formulation may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

A polar co-solvent such as C₂₋₆ aliphatic alcohols and polyols e.g. ethanol, isopropanol and propylene glycol, preferably ethanol, may be included in the MDI aerosol formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise the part or all of the medicament component, such formulations being commonly referred to as solution formulations.

A surfactant may also be employed in the MDI aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio.

The final MDI aerosol formulation desirably contains 0.005-10% w/w, preferably 0.005 to 5% w/w, especially 0.01 to 1.0% w/w, of medicament relative to the total weight of the formulation.

The medicament dispenser is one aspect, a dry powder inhaler type dispenser arranged to dispense medicament in dry powder form. In a particular aspect, the dispenser is of the ‘active DPI’ type and includes a source of compressed air to assist in aerosolising the dry powder form medicament dose.

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

The medicament dispenser is one aspect, arranged to dispense medicament in fluid form. In this aspect, the medicament is formulated as any suitable fluid formulation, particularly a solution (e.g. aqueous) formulation or a suspension formulation, optionally containing other pharmaceutically acceptable additive components.

Suitable formulations (e.g. solution or suspension) may be stabilised (e.g. using hydrochloric acid or sodium hydroxide) by appropriate selection of pH. Typically, the pH will be adjusted to between 4.5 and 7.5, preferably between 5.0 and 7.0, especially around 6 to 6.5.

Suitable formulations (e.g. solution or suspension) may comprise one or more excipients. By the term “excipient”, herein, is meant substantially inert materials that are nontoxic and do not interact with other components of a composition in a deleterious manner including, but not limited to, pharmaceutical grades of carbohydrates, organic and inorganic salts, polymers, amino acids, phospholipids, wetting agents, emulsifiers, surfactants, poloxamers, pluronics, and ion exchange resins, and combinations thereof.

Suitable carbohydrates include monosaccharides include fructose; disaccharides, such as, but not limited to lactose, and combinations and derivatives thereof; polysaccharides, such as, but not limited to, cellulose and combinations and derivatives thereof; oligosaccharides, such as, but not limited to, dextrins, and combinations and derivatives thereof; polyols, such as but not limited to sorbitol, and combinations and derivatives thereof.

Suitable organic and inorganic salts include sodium or calcium phosphates, magnesium stearate, and combinations and derivatives thereof.

Suitable polymers include natural biodegradable protein polymers, including, but not limited to, gelatin and combinations and derivatives thereof; natural biodegradable polysaccharide polymers, including, but not limited to, chitin and starch, crosslinked starch and combinations and derivatives thereof; semisynthetic biodegradable polymers, including, but not limited to, derivatives of chitosan; and synthetic biodegradable polymers, including, but not limited to, polyethylene glycols (PEG), polylactic acid (PLA), synthetic polymers including but not limited to polyvinyl alcohol and combinations and derivatives thereof;

Suitable amino acids include non-polar amino acids, such as leucine and combinations and derivatives thereof. Suitable phospholipids include lecithins and combinations and derivatives thereof.

Suitable wetting agents, surfactants and/or emulsifiers include gum acacia, cholesterol, fatty acids including combinations and derivatives thereof. Suitable poloxamers and/or Pluronics include poloxamer 188, Pluronic® F-108, and combinations and derivations thereof. Suitable ion exchange resins include amberlite IR120 and combinations and derivatives thereof;

Suitable solution formulations may comprise a solubilising agent such as a surfactant. Suitable surfactants include α-[4-(1,1,3,3-tetramethylbutyl)phenyl]-ω-hydroxypoly(oxy-1,2-ethanediyl) polymers including those of the Triton series e.g. Triton X-100, Triton X-114 and Triton X-305 in which the X number is broadly indicative of the average number of ethoxy repeating units in the polymer (typically around 7-70, particularly around 7-30 especially around 7-10) and 4-(1,1,3,3-tetramethylbutyl)phenol polymers with formaldehyde and oxirane such as those having a relative molecular weight of 3500-5000 especially 4000-4700, particularly Tyloxapol. The surfactant is typically employed in a concentration of around 0.5-10%, preferably around 2-5% w/w based on weight of formulation.

Suitable solution formulations may also comprise hydroxyl containing organic co-solvating agents include glycols such as polyethylene glycols (eg PEG 200) and propylene glycol; sugars such as dextrose; and ethanol. Dextrose and polyethylene glycol (eg PEG 200) are preferred, particularly dextrose. Propylene glycol is preferably used in an amount of no more than 20%, especially no more than 10% and is most preferably avoided altogether. Ethanol is preferably avoided. The hydroxyl containing organic co-solvating agents are typically employed at a concentration of 0.1-20% e.g. 0.5-10%, e.g. around 1-5% w/w based on weight of formulation.

Suitable solution formulations may also comprise solublising agents such as polysorbate, glycerine, benzyl alcohol, polyoxyethylene castor oils derivatives, polyethylene glycol and polyoxyethylene alkyl ethers (e.g. Cremophors, Brij).

Suitable solution formulations may also comprise one or more of the following components: viscosity enhancing agents; preservatives; and isotonicity adjusting agents.

Suitable viscosity enhancing agents include carboxymethylcellulose, veegum, tragacanth, bentonite, hydroxypropylmethylcellulose, hydroxypropylcellulose, hydroxyethylcellulose, poloxamers (eg. poloxamer 407), polyethylene glycols, alginates xanthym gums, carageenans and carbopols.

Suitable preservatives include quaternary ammonium compounds (e.g. benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium edetate (EDTA) and other anti-microbial agents such as chlorhexidine, chlorocresol, sorbic acid and its salts and polymyxin.

Suitable isotonicity adjusting agents act such as to achieve isotonicity with body fluids (e.g. fluids of the nasal cavity), resulting in reduced levels of irritancy associated with many nasal formulations. Examples of suitable isotonicity adjusting agents are sodium chloride, dextrose and calcium chloride.

Suitable suspension formulations comprise an aqueous suspension of particulate medicament and optionally suspending agents, preservatives, wetting agents or isotonicity adjusting agents.

The particulate medicament suitably has a mass mean diameter (MMD) of less than 20 μm, preferably between 0.5-10 μm, especially between 1-5 μm. If particle size reduction is necessary, this may be achieved by techniques such as micronisation and/or microfluidisation.

Suitable suspending agents include carboxymethylcellulose, veegum, tragacanth, bentonite, methylcellulose and polyethylene glycols.

Suitable wetting agents function to wet the particles of medicament to facilitate dispersion thereof in the aqueous phase of the composition. Examples of wetting agents that can be used are fatty alcohols, esters and ethers. Preferably, the wetting agent is a hydrophilic, non-ionic surfactant, most preferably polyoxyethylene (20) sorbitan monooleate (supplied as the branded product Polysorbate 80).

Suitable preservatives and isotonicity adjusting agents are as described above in relation to solution formulations.

The medicament dispenser device herein is in one aspect, suitable for dispensing fluid medicament formulations for the treatment of inflammatory and/or allergic conditions of the nasal passages such as rhinitis e.g. seasonal and perennial rhinitis as well as other local inflammatory conditions such as asthma, COPD and dermatitis.

One suitable nasal dosing regime would be for the patient to inhale slowly through the nose subsequent to the nasal cavity being cleared. During inhalation the formulation would be applied to one nostril while the other is manually compressed. This procedure would then be repeated for the other nostril. Typically, one or two inhalations per nostril would be administered by the above procedure up to three times each day, ideally once daily. Each dose, for example, may deliver 5 μg, 50 μg, 100 μg, 200 μg or 250 μg of active medicament. The precise dosage is either known or readily ascertainable by those skilled in the art.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

What is claimed is:

1. A medicament dispenser device comprising
a housing including a dispensing outlet;
a medicament discharge device moveably housed within the housing, the medicament discharge device comprising a medicament container for storing the medicament to be dispensed and a medicament dispensing mechanism for dispensing medicament from the container to the outlet;
a user operable actuator moveable with respect to the medicament discharge device to apply an actuating force to the dispensing mechanism; and
an actuation indicator responsive to application of said actuating force,
wherein a pre-load means is provided to said user operable actuator to prevent application of the actuating force to the dispensing mechanism and actuation indicator until a pre-determined threshold force is applied to the user operable actuator.

2. A medicament dispenser device according to claim 1, wherein the dispensing outlet is in the form of a mouthpiece for insertion into an oral cavity.

3. A medicament dispenser device according to claim 1, wherein the dispensing outlet is in the form of a nozzle for insertion into a nasal cavity.

4. A medicament dispenser device according to claim 1, wherein the medicament discharge device comprises an aerosol medicament container suitable for containing a propellant-based aerosol medicament formulation.

5. A medicament dispenser device according to claim 4, wherein the dispensing mechanism comprises a metering valve.

6. A medicament dispenser device according to claim 1, wherein the medicament discharge device comprises a pump dispenser for pumped dispensing of medicament in fluid form.

7. A medicament dispenser device according to claim 6, wherein the dispensing mechanism comprises a compression pump.

8. A medicament dispenser device according to claim 1, wherein the medicament discharge device comprises a syringe dispenser for injection of medicament in fluid form.

9. A medicament dispenser device according to claim 1, wherein the user operable actuator is moveable transversely with respect to a longitudinal axis defined by the medicament discharge device to apply an actuating force directly or indirectly to the medicament container.

10. A medicament dispenser device according to claim 1, wherein the user operable actuator is arranged to apply mechanical advantage.

11. A medicament dispenser device according to claim 10, wherein the user operable operator has a form selected from the group consisting of lever, cam and screw form.

12. A medicament dispenser device according to claim 10, wherein the user operable actuator comprises at least one lever pivotally connecting to the housing.

13. A medicament dispenser device according to claim 12, wherein the lever acts on a transition piece connecting to a neck of the medicament container.

14. A medicament dispenser device according to claim 13, wherein the transition piece is in the form of a collar.

15. A medicament dispenser device according to claim 1, wherein the actuation indicator includes a display for displaying dose count information.

16. A medicament dispenser device according to claim 1, wherein the actuation indicator comprises an actuation sensor.

17. A medicament dispenser device according to claim 16, wherein the actuation sensor is sensitive to a stimulus selected from the group consisting of electro magnetic radiation, magnetic field, light, motion, temperature, pressure, sound, oxygen concentration, carbon dioxide concentration and moisture.

18. A medicament dispenser device according to claim 16, wherein the actuation indicator associates mechanically or electronically with the actuation sensor.

19. A medicament dispenser device according to claim 1, wherein the pre-load means is physically interposed between the user operable actuator and the medicament container.

20. A medicament dispenser device according to claim 19, wherein the pre-load means may comprises a step formed on the medicament container wherein the step is over-ridden when the pre-determined threshold force is applied to the user operable actuator.

21. A medicament dispenser device according to claim 19, wherein the pre-load means comprises a step formed on the user operable actuator wherein the step is over-ridden when the pre-determined threshold force is applied to the user operable actuator.

22. A medicament dispenser device according to claim 19, wherein the pre-load means comprises at least one detent formed on one of the medicament container or the user operable actuator and a recess formed on the other of the medicament container or the user operable actuator wherein the or each detent rides out of the recess when the pre-determined threshold force is applied to the user operable actuator.

23. A medicament dispenser device according to claim 1, wherein the pre-load means is interposed between the housing and the medicament container.

24. A medicament dispenser device according to claim 23, wherein the pre-load means comprises one or more detents formed on the medicament container for engagement with part of the housing, the or all of the detents being disengageable from the housing when the pre-determined threshold force is applied to the user operable actuator.

25. A medicament dispenser device according to claim 23, wherein the pre-load means comprises one or more detents formed on the housing for engagement with part of the medicament container, the or all of the detents being disengageable from the medicament container when the pre-determined threshold force is applied to the user operable actuator.

26. A medicament dispenser device according to claim 1, wherein the pre-load means is interposed between the housing and the user operable actuator.

27. A medicament dispenser device according to claim 26, wherein the pre-load means comprises at least one detent formed on the housing for engagement with the user operable actuator, the or all of the detents being disengageable from the user operable actuator when the pre-determined threshold force is applied to the user operable actuator.

28. A medicament dispenser device according to claim 26, wherein the pre-load means comprises at least one detent formed on the user operable actuator for engagement with part of the housing, the or all of the detents being disengageable from the housing when the pre-determined threshold force is applied to the user operable actuator.

29. A medicament dispenser device according to claim 1, wherein the pre-load means defines a variable mechanical ratio.

30. A medicament dispenser device according to claim 29, wherein said variable mechanical ratio is defined by the profile of interaction of a surface of the user operable actuator with a follower element provided to the container or a fitting provided thereto.

31. A medicament dispenser device according to claim 29, wherein said variable mechanical ratio is defined by the profile of interaction of a surface of the container or a fitting provided thereto with a follower element provided to the user operable actuator.

32. A medicament dispenser according to claim 30, wherein the variable mechanical ratio defines a ‘two step’ profile characterized by an initial high gradient and a subsequent lower gradient.

33. A medicament dispenser device according to claim 32, wherein the high and lower gradient profiles are linear.

34. A medicament dispenser device according to claim 32, wherein the high and lower gradient profiles are curved and have a gradual break point therebetween.

35. A medicament dispenser device according to claim 34, wherein the high and lower gradient profiles have part-circle forms.

36. A medicament dispenser device according to claim 1, additionally comprising force modifying means for modifying the force applied to the container by the finger operable means.

37. A medicament dispenser device according to claim 36, in which said force modifying means amplifies the force applied to the container by the user operable actuator.

38. A medicament dispenser device according to claim 37, in which the amplification is provided in a uniform manner.

39. A medicament dispenser device according to claim 38, in which the degree of amplification is from 1.5 to 10.

40. A medicament dispenser device according to claim 36, in which the force modifying means is integral with the user operable actuator.

41. A medicament dispenser device according to claim 36, in which the force modifying means is located between the user operable actuator and the container.

42. A medicament dispenser device according to claim 36, in which the force modifying means comprises a lever, cam or screw element.

43. A medicament dispenser device according to claim 36, in which the force modifying means acts once the pre-determined force has been applied to the finger operable means.

44. A medicament dispenser device according to claim 43, in which the force modifying means acts such that once the predetermined force has been applied to the finger operable means the modified force applied to the container is relatively constant.

45. A medicament dispenser device according to claim 43, in which the force modifying means acts such that once the pre-determined force has been applied to the finger operable means the modified force applied to the container increases on a relatively constant basis.

46. A medicament dispenser device according to claim 36, in which the force modifying means additionally comprises a stop element.

47. A housing assembly for reversible receipt of a medicament discharge device, said medicament discharge device comprising a medicament container for storing the medicament to be dispensed and a medicament dispensing mechanism for dispensing medicament from the container, the housing assembly comprising
a housing including a dispensing outlet for insertion into a body cavity;
a user operable actuator moveable with respect to the medicament discharge device to apply an actuating force to the dispensing mechanism; and
an actuation indicator responsive to application of said actuating force,
wherein a pre-load means is provided to said user operable actuator to prevent application of the actuating force to the dispensing mechanism and actuation indicator until a pre-determined threshold force is applied to the user operable actuator.

48. A kit of parts comprising a housing assembly according to claim 47; and a medicament discharge device receivable thereby.

49. A medicament dispenser device comprising
a housing including a dispensing outlet for insertion into a body cavity;
within the housing, a medicament release device, the medicament release device comprising a medicament container for storing the medicament to be dispensed and a medicament release mechanism for releasing medicament from the container to a release position within the housing;
an airflow generator moveably housed within the housing, said airflow generator means capable on actuation, of providing airflow to said release position for aerosolising said released medicament;
a user operable actuator moveable to apply an actuating force to the airflow generator; and
an actuation indicator responsive to application of said actuating force,
wherein a pre-load means is provided to said user operable actuator to prevent application of the actuating force to the airflow generator and actuation indicator until a pre-determined threshold force is applied to the user operable actuator.
